(19) United States
(12) Patent Application Publication
(10) Pub. No.: US 2024/0186018 A1
(43) Pub. Date: Jun. 6, 2024

(54) NEURAL POINT PROCESS-BASED EVENT PREDICTION FOR MEDICAL DECISION MAKING

(71) Applicant: NEC Laboratories America, Inc., Princeton, NJ (US)

(72) Inventors: Yuncong Chen, Plainsboro, NJ (US); Haifeng Chen, West Windsor, NJ (US); Zhengzhang Chen, Princeton Junction, NJ (US); Yanchi Liu, Monmouth Junction, NJ (US); LuAn Tang, Cranbury, NJ (US)

(21) Appl. No.: 18/493,331

(22) Filed: Oct. 24, 2023

Related U.S. Application Data
(60) Provisional application No. 63/419,003, filed on Oct. 25, 2022.

(51) Int. Cl. G16H 50/30

(52) U.S. Cl. CPC G16H 50/30

(57) ABSTRACT

Methods and systems for event prediction include encoding a multivariate time series and a multi-type event sequence using respective transformers and an aggregation network to generate a feature vector. Event prediction is performed using the feature vector to identify a next event to occur within a system. A corrective action is performed responsive to the next event to prevent or mitigate an effect of the next event. The predicted next event can be used in a healthcare context to support decision making by medical professionals with respect to the treatment of a patient. The encoding may include machine learning models to implement the transformers and the aggregation network using deep learning.

### RELATED APPLICATION INFORMATION

This application claims priority to U.S. Patent Application No. 63/419,003, filed on Oct. 25, 2022, incorporated herein by reference in its entirety.

### BACKGROUND

### Technical Field

The present invention relates to event prediction and, more particularly, to inferring system conditions from event history and time series information.

### Description of the Related Art

Event prediction helps to manage complex systems. In cyber-physical systems, such as information technology systems, hardware failures may be predicted and preventative maintenance can be planned. In healthcare systems, adverse event prediction can help doctors adjust treatments early to prevent negative health outcomes.

### SUMMARY

A method for event prediction includes encoding a multivariate time series and a multi-type event sequence using respective transformers and an aggregation network to generate a feature vector. Event prediction is performed using the feature vector to identify a next event to occur within a system. A corrective action is performed responsive to the next event to prevent or mitigate an effect of the next event.

A system for event prediction includes a hardware processor and a memory that stores a computer program. When executed by the hardware processor, the computer program causes the hardware processor to encode a multivariate time series and a multi-type event sequence using respective transformers and an aggregation network to generate a feature vector, to perform event prediction using the feature vector to identify a next event to occur within a system, and to perform a corrective action responsive to the next event to prevent or mitigate an effect of the next event.

A method for performing a treatment includes measuring time series information relating to a patient. The time series information and a health event sequence for the patient are encoded using respective transformers and an aggregation network to generate a feature vector. Event prediction is performed using the feature vector to identify a next health event to occur within a system. A corrective action is performed responsive to the next health event to prevent or mitigate a negative health effect of the next health event.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram of a cyber-physical system that implements event prediction based on time series information and event log information, in accordance with an embodiment of the present invention;

FIG. 2 is a diagram of a patient in a healthcare context receiving treatment in accordance with a predicted health event, in accordance with an embodiment of the present invention;

FIG. 3 is a block diagram of a healthcare facility where information on the functioning of treatment systems and medical records are used to predict events, in accordance with an embodiment of the present invention;

FIG. 4 is a block/flow diagram of an event prediction method/system, in accordance with an embodiment of the present invention;

FIG. 5 is a block/flow diagram of a method for training and using a deep learning model for event prediction, in accordance with an embodiment of the present invention;

FIG. 6 is a block/flow diagram of a method of training a deep learning model to predict events, in accordance with an embodiment of the present invention;

FIG. 7 is a block diagram of a computing device that can train and use an event prediction model to predict and correct events, in accordance with an embodiment of the present invention;

FIG. 8 is a diagram of an exemplary neural network architecture that may be used to implement part of an event prediction model, in accordance with an embodiment of the present invention; and

FIG. 9 is a diagram of an exemplary deep neural network architecture that may be used to implement part of an event prediction model, in accordance with an embodiment of the present invention.

### DETAILED DESCRIPTION OF PREFERRED EMBODIMENTS

Event prediction makes use of historical event information, but may also make use of information about system conditions that are constantly changing. System conditions can be inferred from current time series information about system metrics.

Events and time series information have complex causal interactions. For example, an increasing trend in memory usage in a computer system is likely to eventually cause an out-of-memory event. On the other hand, an elevated level of disk queue length can be the result of a disk-access-intensive application launch event. Event prediction that considers time series information as well as event history produces better outcomes than systems that consider either individually.

Due to the volatility of dynamic processes in complex systems, a probability distribution may be more effective than point-wise next-event estimation. Furthermore, explainability of a prediction result can be challenging. The past events and metric conditions that cause a predicted event may be identified to aid decision-making in improving future performance.

Toward that end, a machine learning model can be used to predict the type and time of a next event, given a history of previous events and one or more time series metrics. Transformers and an attention mechanism are used to explicitly model the interaction between system events and metrics, encapsulating the interactions in hidden states. A point process may be used to condition on the hidden states, to generate probabilistic distributions of the type and time of a next event. This may be accomplished in several ways, such as modeling an intensity function and modeling a density function.

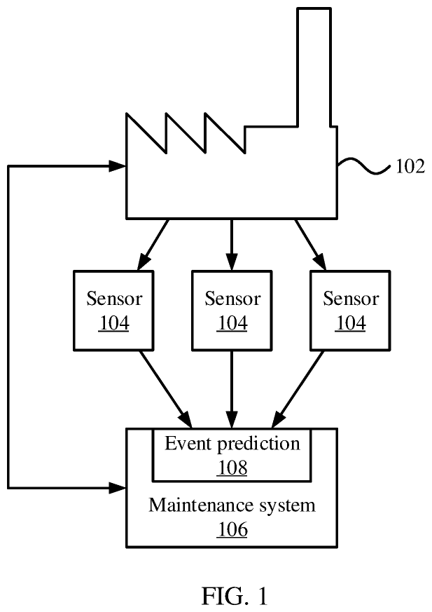

Referring now to FIG. 1, a maintenance system 106 in the context of a monitored system 102 is shown. The monitored system 102 can be any appropriate system, including physical systems such as manufacturing lines and physical plant operations, electronic systems such as computers or other computerized devices, software systems such as operating systems and applications, and cyber-physical systems that combine physical systems with electronic systems and/or software systems. Exemplary systems 102 may include a wide range of different types, including railroad systems, power plants, vehicle sensors, data centers, and transportation systems.

One or more sensors 104 record information about the state of the monitored system 102. The sensors 104 can be any appropriate type of sensor including, for example, physical sensors, such as temperature, humidity, vibration, pressure, voltage, current, magnetic field, electrical field, and light sensors, and software sensors, such as logging utilities installed on a computer system to record information regarding the state and behavior of the operating system and applications running on the computer system. The information generated by the sensors 104 can be in any appropriate format and can include sensor log information generated with heterogeneous formats.

The sensors 104 may transmit the logged sensor information to an anomaly maintenance system 106 by any appropriate communications medium and protocol, including wireless and wired communications. The maintenance system 106 can, for example, identify abnormal or anomalous behavior by monitoring the multivariate time series that are generated by the sensors 104. Once anomalous behavior has been detected, the maintenance system 106 communicates with a system control unit to alter one or more parameters of the monitored system 102 to correct the anomalous behavior.

Exemplary corrective actions include changing a security setting for an application or hardware component, changing an operational parameter of an application or hardware component (for example, an operating speed), halting and/or restarting an application, halting and/or rebooting a hardware component, changing an environmental condition, changing a network interface's status or settings, etc. The maintenance system 106 thereby automatically corrects or mitigates the anomalous behavior. By identifying the particular sensors 104 that are associated with the anomalous classification, the amount of time needed to isolate a problem can be decreased.

Each of the sensors 104 outputs a respective time series, which encodes measurements made by the sensor over time. For example, the time series may include pairs of information, with each pair including a measurement and a timestamp, representing the time at which the measurement was made. Each time series may be divided into segments, which represent measurements made by the sensor over a particular time range. Time series segments may represent any appropriate interval, such as one second, one minute, one hour, or one day. Time series segments may represent a set number of collection time points, rather than a fixed period of time, for example covering 100 measurements.

The maintenance system 106 may track occurrences of events relating to the condition of the monitored system 102. For example, the maintenance system 106 may receive information relating to workload, starting and stopping of jobs, and malfunctions. This information may be recorded with appropriate time-stamp and other condition information relating to the state of the monitored system 102 at the time the event occurred, for example including information collected by the sensors 104. Whereas time series information may be recorded periodically or on an aperiodic but frequent basis, the event information may be recorded whenever a discrete event occurs.

The maintenance system 106 may predict when a future event occur using event prediction 108. The event prediction makes use of both the event history information and the time series information to determine when an event is likely to occur. The maintenance system 106 can furthermore determine which source(s) of information contribute most to the prediction, so that a corrective action taken by the maintenance system 106 can be tailored to a root cause of a problem.

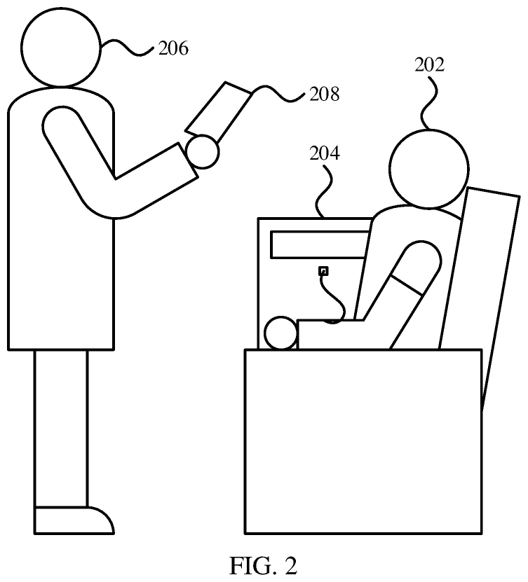

Referring now to FIG. 2, patient 202 is shown in the context of a healthcare system. For example, the patient 202 may be in a hemodialysis (also known simply as “dialysis”) session. During dialysis, a dialysis machine 204 automatically draws the patient's blood, processes and purifies the blood, and then reintroduces the purified blood to the patient's body. Dialysis can take as long as four hours to complete, and may be performed every three days, though other durations and periods are contemplated. Although dialysis is specifically contemplated, it should be understood that any appropriate medical procedure or monitoring may be used instead.

Before, during, and after a dialysis session, a patient 202 may experience a health event relating to the treatment. Such health events can be dangerous to the patient 202, but can be predicted based on knowledge of previous health events and the patient's present health metrics. Before the patient 202 undergoes a medical procedure or treatment, such as dialysis, a medical professional 206 reviews a recommendation 208 that includes a prediction score. This prediction score indicates a likelihood that a health event will occur during the dialysis session. The recommendation 208 may furthermore include information relating to the type of event that is predicted, as well as measurements of the patient's status It is specifically contemplated that this recommendation may be made before the dialysis session begins, so that treatment can be adjusted.

The recommendation may be made based on a variety of input information. Part of that information includes a static profile of the patient, for example including information such as age, sex, starting time of dialysis, previous health events, etc. The information also includes dynamic data, such as dialysis measurement records, which may be taken at every dialysis session, blood pressure, weight, venous pressure, blood test measurements, and cardiothoracic ratio (CTR). The blood test measurements may be taken regularly, for example at a frequency of twice per month, and may measure such factors as albumin, glucose, and platelet count. The CTR may also be taken regularly, for example at a frequency of once per month. Dynamic information may also be recorded during the dialysis session, for example using sensors in the dialysis machine 204. The dynamic information may be modeled as time series over their respective frequencies.

In addition, the systems themselves may be monitored within a healthcare environment. For example, operational parameters of a dialysis machine 204 or any other system in a hospital or other healthcare facility many be monitored, along with a history of past events at the system, to predict events as described below. When an event is predicted, a corrective action may be performed and/or a system administrator may be notified.

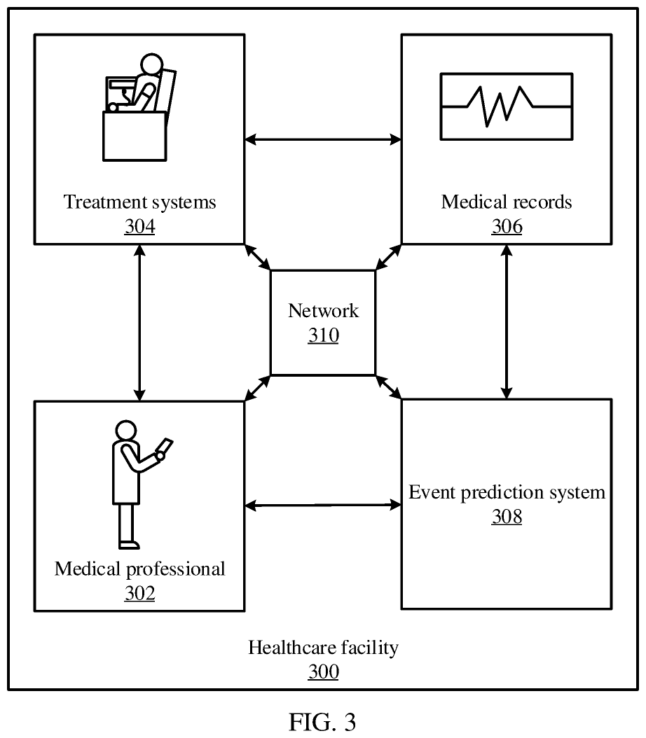

Referring now to FIG. 3, a diagram of event prediction in the context of a healthcare facility 300. Rather than, or in addition to, predicting events in the context of a single patient's treatments, the present principles may be applied to all of the systems within the facility. This may be used to help monitor and treat multiple patients, for example responding to changes in environmental conditions and shortages of materials. Such facilities may also be vulnerable to cyber-attack, and predicting events in such a context can help to identify and stop the attack, preserving the facility's ability to treat patients.

The healthcare facility may include one or more medical professionals 302 who provide information relating to events and measurements of system status to an event prediction system 308. Treatment systems 304 may furthermore monitor patient status to generate medical records 306 and may be designed to automatically administer and adjust treatments as needed.

Based on information drawn from at least the medical professionals 302, treatment systems 304, and medical records 306, event prediction system 308 can predict events and can automatically respond to prevent or mitigate the predicted events. For example, corrective action can be taken and/or facility administrators may be notified. In the event that an administrator is notified, the event prediction can be used to support decision-making by the hospital's administrator.

The different elements of the healthcare facility 300 may communicate with one another via a network 310, for example using any appropriate wired or wireless communications protocol and medium. Thus the output of the event prediction system 308 may access remotely stored medical records 306, may communicate with the treatment systems 304, and may receive instructions and send reports to medical professionals 302.

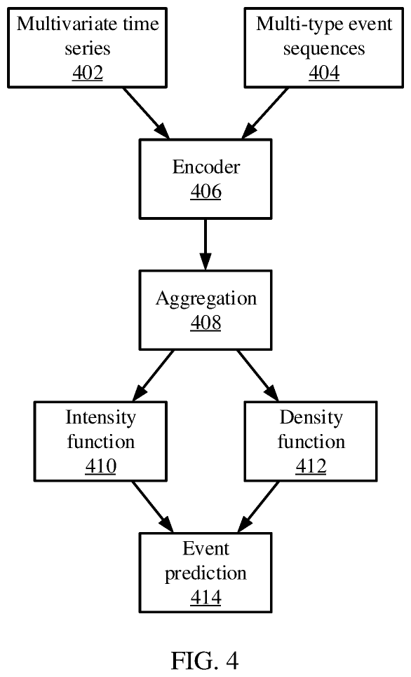

Referring now to FIG. 4, event prediction using time series information and event sequence information is shown. The input may include multivariate time series 402, for example combining multiple time series from different respective sensors. The input may further include multi-type event sequences 404, which may indicate a type of event, a time that the event occurred, and any other appropriate information relating to the event. The multi-type event sequences may include multiple distinct types of event.

An encoder 406 embeds the inputs into a latent space, generating a set of features that represent the joint inputs 402 and 404. Each input may generate a respective feature vector, and these vectors may be aggregated together 408 to create a context vector. The encoder(s) 406 may include transformers with stacks of self-attention and cross-attention layers that fuse the information among different time steps from each sequence. For the encoder of the time series information, the input sequence may be a concatenation of a timestamp embedding and embedded metric value vectors. For encoder of the event sequence information, the input may be a concatenation of the embeddings representing various data of the log events (e.g., template type embedding vector, parameter embedding vector, and time embedding vector). The hidden states from each time step of each sequence are then concatenated and passed to the aggregator network. 408. The aggregator network 408 may include self-attention layers that fuse the information from all time steps of both streams and outputs a context vector. The aggregator network 408 accepts a concatenation of the two sequences of output states from the encoder(s) 406.

The context vector may be used as an input to one or more of an intensity function 410 and a density function 412. The output of the intensity function 410 and/or the density function 412 is an event prediction 414.

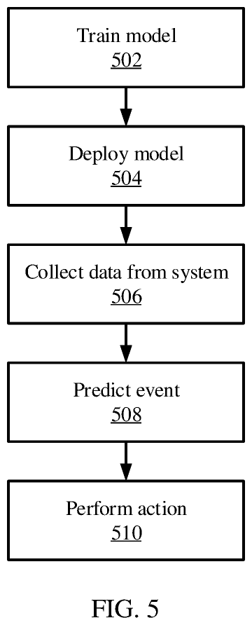

Referring now to FIG. 5, a method for predicting and proactively addressing events is shown. Block 502 acquires a set of training data, which may include system metric time series and timestamped event sequences, and trains a machine learning model, as will be described in greater detail below, to generate event predictions. The trained model is then deployed 504 to an installation, for example in the management system 106 of a cyber-physical system or a healthcare analytics system.

New data may be collected from the installation, including event information and multivariate time series information, at block 506. This new data represents the operational state of the system and its particular event history. The trained model is used in block 508 to predict an event relating to the system. For example, this event prediction may relate to an expected system malfunction or adverse health event.

Block 510 then performs an action to prevent or mitigate the harm of the predicted event. In the context of cyber-physical system 102, the action may include performing an automated response that addresses one or more sub-systems that are expected to be related to the predicted event. For example, the action may include changing an environmental parameter to prevent overheating or may shut down a sub-system to prevent damage.

In the context of a patient 202, where the predicted event is an adverse health event, the action may include an automatic adjustment to a treatment, for example adjusting the operation of a dialysis machine 204, adjusting dosage of an intravenously administered drug, or halting a treatment that is deemed to be dangerous.

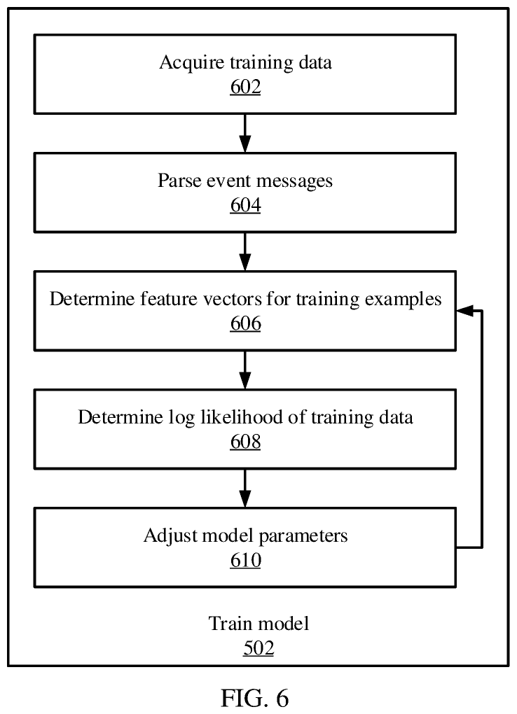

Referring now to FIG. 6, additional detail on training 502 is shown. Block 602 acquires a set of training data, including synchronized system metric time series and timestamped system event sequences. Block 604 parses the event messages in the system event sequences to identify the event type from each message, for example representing each distinct type of event as a different integer value.

The training data may be split into context windows, for example using overlapping windows of a fixed length. Each training sample may come from a respective time window, including the time series segment xᵢ in the window, the event subsequence vᵢ in the window, and the event (type uᵢ at time tᵢ) that follows immediately after the window.

Block 606 determines feature vectors for training examples. The training examples may be sampled from the training data as appropriate, and the feature vectors may be computed using the encoder 406 and aggregation network 408. The encoder may include two separate transformer networks, one for the multivariate time series and the other for the event sequence. A shared time embedding layer may be learned using a time-to-vector encoder that converts timestamps to vectors.

The multivariate time series may first be processed by a one-dimensional convolutional layer, with the result at each timestamp being concatenated with the corresponding time embedding vector before being input to the encoder 406. Event sequences may be first parsed by a log parser to decompose each event message into a template and parameters. For example, the message, “ESMCommonService has transitioned to the stopped state,” may be converted to a template, “[*] has transitioned to the stopped state,” and the parameters, “ESMCommonService.” A template type embedding layer and a parameter embedding layer are learned to convert the template type and parameters to vectors, respectively. For each event in the sequence, the template type embedding vector, the parameter embedding vector, and the time embedding vector are concatenated and used as input to the transformer encoder.

The aggregation network 408 is a stack of self-attention layers. The hidden state of the time series transformer encoder at the last timestep may be used to compute a latent vector h. The latent vector h is then used to condition a neural point process.

Block 608 determines a log likelihood of the training example using, e.g., the intensity function 410 or the density function 412, modeled by the neural point process. The model parameters are adjusted 610 using, e.g., a stochastic gradient descent, to maximize the log likelihood of true next events in terms of both type and time. This process may be repeated until the model parameters converge.

The determination of the log likelihood in block 608 depends on the modeling function that is used. An intensity function 410 uses the conditional intensity functions λᵤ(t) for each event type u and time t until a next event. An intensity function represents the expected number of events at an infinitesimally small time period and may determine the probability of the time and type of the next event. The function may be computed by a neural network that is conditioned on context vector h t and a linear function of the time from the last observed i with a softplus activation function:

λₖ=softplus(Wₖhᵢ+αₖ(t−tᵢ)

where Wₖ and αₖ are learnable parameters and k denotes an event type. The parameter αₖ controls whether the intensity of an event increases or decreases with time and Wₖ denotes the learnable weights of a neural network. The negative log likelihood for the training data may then be computed. For the s^{th} sequence with nₛ events {(kᵢ,tᵢ),i=1 . . . nₛ}, the negative log likelihood using the intensity function 410 is:

$${NLL}_{int}^{s} = {\sum }_{i = 1}^{{n}_{s}} - log {λ}_{{k}_{i}}^{*} ( {t}_{i} ) + {\sum }_{k = 1}^{K} {\int }_{{t}_{i - 1}}^{{t}_{i}} {λ}_{k}^{*} ( {t}^{′} ) {dt}^{′}$$

where λ*ₖ(t)=λₖ(t| [symbol] ₜ) is the conditional intensity function for event type k and [symbol] ₜ={(kᵢ,tᵢ)tᵢ≤t} is the history up to time t. The final loss using the intensity function 410 over all S sequences is:

$${ℒ}_{int} = {\sum }_{s = 1}^{S} {NLL}_{int}^{s}$$

Block 608 may instead use a density function 412 to model the time probability and event type probability independently. The time probability is modeled by estimating the conditional density function instead of the intensity function. The density distribution p(τ) is the probability of a next event happening a time r after the last event. It is designed as a mixture of log-normal distributions, following:

$$p ( τ \vert w , μ , s ) = {\sum }_{k = 1}^{K} {w}_{k} \cdot \frac{1}{τ {s}_{k} \sqrt{2 π}} \cdot {e}^{- \frac{{( log τ - {μ}_{k} )}^{2}}{2 {s}_{k}^{2}}}$$

where wₖ are component weights of w, μₖ are component locations of μ, and sₖ are scales. The scale of a log-normal component in a mixture distribution is the width of that component. It can be understood as representing the uncertainty of the component. These parameters are generated by a neural network conditioned on the context vector hₜ:

wₖ=softmax(U_{w}hᵢ+b_{w})

sₖ=e\hat{ }(Uₛhᵢ+bₛ)

μₖ=U_{μ}hᵢ+b_{μ}

where U_{w}, Uₛ, U_{μ}, b_{w}, bₛ, and b_{μ} are learnable parameters and s in this context is a learnable vector parameter.

The type probability may be expressed as a categorical distribution given by another neural network conditioned on hᵢ. The output of the softmax activation is a vector, where the u^{th} element p(u) is the probability that the next event is of type u. The formula is:

p(u)=softmax(V² tanh(V_{π}¹hᵢ+b_{π}¹)+b_{π}²)

where V_{π}¹, V_{π}², b_{π}¹, and b_{π}² are learnable parameters. The negative log likelihood for the density function can be determined as:

$${NLL}_{den}^{s} = - {\sum }_{i = 2}^{{n}_{s}} log p ( {t}_{i} - {t}_{i - 1} ) + log p ( {k}_{i} )$$

The final loss for the density function may be given as

$${ℒ}_{d e n} = {\sum }_{s = 1}^{S} {NLL}_{d e n}^{s}$$

As above, the encoder 406, aggregation network 408, and neural point process parameters may be trained together using stochastic gradient descent to minimize the negative log likelihood.

After the model is trained and deployed, at a time t, the events and time series in a fixed-size context window preceding t may be input to the model. The model outputs the intensity function or density function value over the type and time of the next event. This information can be used to assist downstream decision-making such as predictive maintenance. If a most probable event time and type prediction is needed, the intensity function or density function can be used to obtain his information.

When the intensity function 410 is used, the expected next event time \hat{t}ⱼ₊₁ and type \hat{k}ⱼ₊₁ may be determined as:

$$p ( t \vert {ℋ}_{t} ) = λ ( t \vert {ℋ}_{t} ) {e}^{- {\int }_{{t}_{j}}^{t} λ ( τ \vert {ℋ}_{τ} ) d τ}$$

$${\hat{t}}_{j + 1} = {\int }_{{t}_{j}}^{∞} t \cdot p ( t \vert {ℋ}_{t} ) dt$$

$${\hat{k}}_{j + 1} = arg {max}_{k} ( \frac{{λ}_{k}^{*} ( {\hat{t}}_{j + 1} )}{{λ}^{*} ( {\hat{t}}_{j + 1} )} )$$

where λ*(t)=Σₖ₌₁^{K}λ*ₖ(t) is the sum of type-wise intensity functions.

When the density function 412 is used, the expected next event time can be computed by a weighted average of component means:

$${\hat{t}}_{j + 1} = {\sum }_{k} {w}_{k} {e}^{{μ}_{k} + {s}_{k}^{2} / 2}$$

The expected next event type can be computed by:

$${\hat{k}}_{j + 1} = arg {max}_{u} p ( u )$$

The events and time series readings may furthermore be ranked for a context window according to their attention weights in the aggregation network 408. The top-ranked results are returned as significant events and readings that most influence the predicted event. This information can help to identify root causes of failure events and negative health outcomes.

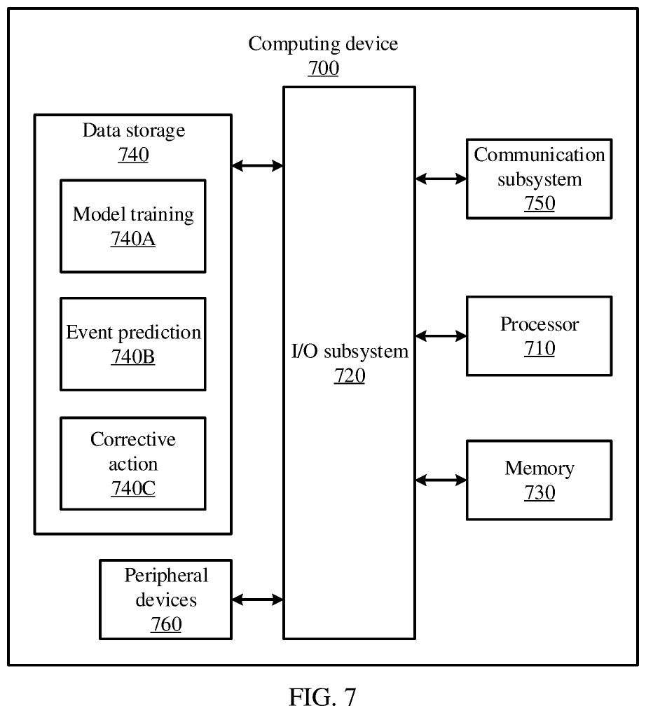

Referring now to FIG. 7, an exemplary computing device 700 is shown, in accordance with an embodiment of the present invention. The computing device 700 is configured to perform event prediction.

The computing device 700 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 700 may be embodied as one or more compute sleds, memory sleds, or other racks, sleds, computing chassis, or other components of a physically disaggregated computing device.

As shown in FIG. 7, the computing device 700 illustratively includes the processor 710, an input/output subsystem 720, a memory 730, a data storage device 740, and a communication subsystem 750, and/or other components and devices commonly found in a server or similar computing device. The computing device 700 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 730, or portions thereof, may be incorporated in the processor 710 in some embodiments.

The processor 710 may be embodied as any type of processor capable of performing the functions described herein. The processor 710 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 730 may be embodied as any type of volatile or non-volatile memory or data storage capable of performing the functions described herein. In operation, the memory 730 may store various data and software used during operation of the computing device 700, such as operating systems, applications, programs, libraries, and drivers. The memory 730 is communicatively coupled to the processor 710 via the I/O subsystem 720, which may be embodied as circuitry and/or components to facilitate input/output operations with the processor 710, the memory 730, and other components of the computing device 700. For example, the I/O subsystem 720 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 720 may form a portion of a system-on-a-chip (SOC) and be incorporated, along with the processor 710, the memory 730, and other components of the computing device 700, on a single integrated circuit chip.

The data storage device 740 may be embodied as any type of device or devices configured for short-term or long-term storage of data such as, for example, memory devices and circuits, memory cards, hard disk drives, solid state drives, or other data storage devices. The data storage device 740 can store program code 740A for training a model, 740B for predicting an event, and/or 740C for performing a corrective action responsive to the predicted event. Any or all of these program code blocks may be included in a given computing system. The communication subsystem 750 of the computing device 700 may be embodied as any network interface controller or other communication circuit, device, or collection thereof, capable of enabling communications between the computing device 700 and other remote devices over a network. The communication subsystem 750 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 700 may also include one or more peripheral devices 760. The peripheral devices 760 may include any number of additional input/output devices, interface devices, and/or other peripheral devices. For example, in some embodiments, the peripheral devices 760 may include a display, touch screen, graphics circuitry, keyboard, mouse, speaker system, microphone, network interface, and/or other input/output devices, interface devices, and/or peripheral devices.

Of course, the computing device 700 may also include other elements (not shown), as readily contemplated by one of skill in the art, as well as omit certain elements. For example, various other sensors, input devices, and/or output devices can be included in computing device 700, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 700 are readily contemplated by one of ordinary skill in the art given the teachings of the present invention provided herein.

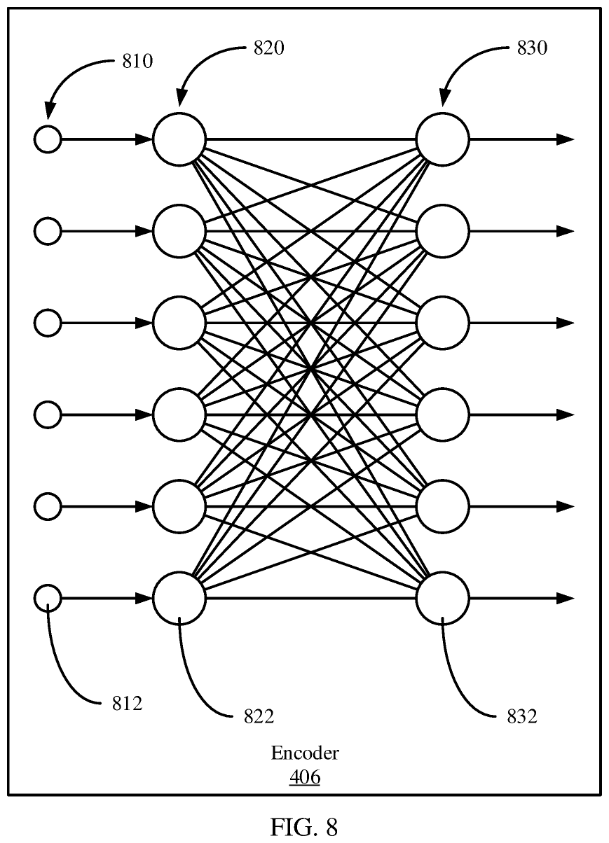

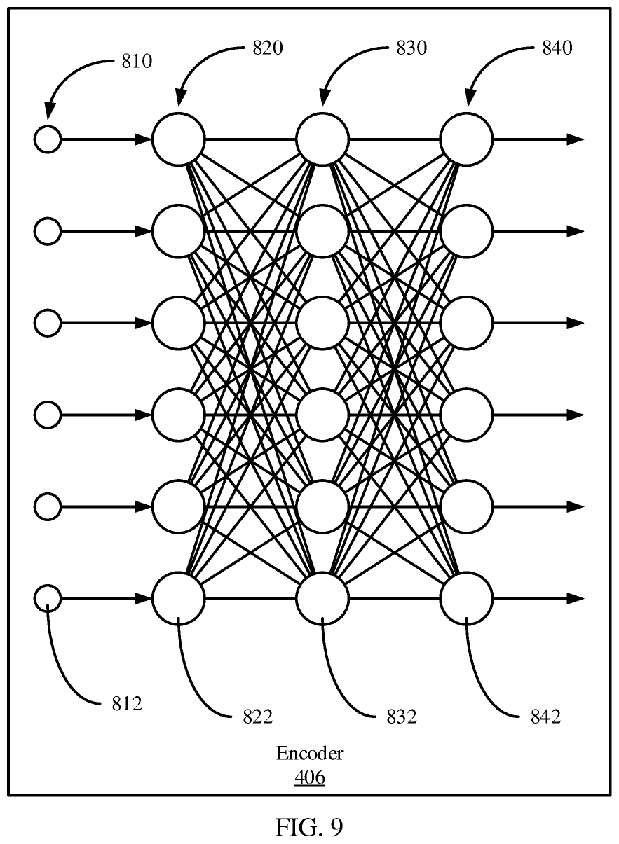

Referring now to FIGS. 8 and 9, exemplary neural network architectures are shown, which may be used to implement parts of the present models, such as the encoder 406. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 820 of source nodes 822, and a single computation layer 830 having one or more computation nodes 832 that also act as output nodes, where there is a single computation node 832 for each possible category into which the input example could be classified. An input layer 820 can have a number of source nodes 822 equal to the number of data values 812 in the input data 810. The data values 812 in the input data 810 can be represented as a column vector. Each computation node 832 in the computation layer 830 generates a linear combination of weighted values from the input data 810 fed into input nodes 820, and applies a non-linear activation function that is differentiable to the sum. The exemplary simple neural network can perform classification on linearly separable examples (e.g., patterns).

A deep neural network, such as a multilayer perceptron, can have an input layer 820 of source nodes 822, one or more computation layer(s) 830 having one or more computation nodes 832, and an output layer 840, where there is a single output node 842 for each possible category into which the input example could be classified. An input layer 820 can have a number of source nodes 822 equal to the number of data values 812 in the input data 810. The computation nodes 832 in the computation layer(s) 830 can also be referred to as hidden layers, because they are between the source nodes 822 and output node(s) 842 and are not directly observed. Each node 832, 842 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 832 in the one or more computation (hidden) layer(s) 830 perform a nonlinear transformation on the input data 812 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for event prediction, comprising:
encoding a multivariate time series and a multi-type event sequence using respective transformers and an aggregation network to generate a feature vector;
performing event prediction using the feature vector to identify a next event to occur within a system; and
performing a corrective action responsive to the next event to prevent or mitigate an effect of the next event.

2. The method of claim 1, wherein performing event prediction uses an intensity function that includes a softplus function of the feature vector and a next arrival time.

3. The method of claim 1, wherein performing event prediction uses a density function that models time probability and type probability independently.

4. The method of claim 1, wherein the transformers and the aggregation network are trained using deep learning, with a set of training data that includes synchronized time series information and timestamped event sequences.

5. The method of claim 1, wherein the aggregation network includes a stack of self-attention layers that convert outputs of the respective transformers to the feature vector.

6. The method of claim 1, wherein a hidden state of the transformer of the multivariate time series is used as a latent vector in the event prediction.

7. The method of claim 1, further determining a ranked list of past events and time series measurements that most influence the predicted event, according to according to attention weights from the aggregation network.

8. The method of claim 1, further comprising reporting the next event to a medical professional to support medical decision-making.

9. The method of claim 1, wherein performing the corrective action includes an action selected from the group consisting of changing a security setting for an application or hardware component, changing an operational parameter of an application or hardware component, halting and/or restarting an application, halting and/or rebooting a hardware component, changing an environmental condition, and changing a network interface's status or settings.

10. A system for event prediction, comprising:
a hardware processor; and
a memory that stores a computer program which, when executed by the hardware processor, causes the hardware processor to:
encode a multivariate time series and a multi-type event sequence using respective transformers and an aggregation network to generate a feature vector;
perform event prediction using the feature vector to identify a next event to occur within a system; and
perform a corrective action responsive to the next event to prevent or mitigate an effect of the next event.

11. The system of claim 10, wherein performing event prediction uses an intensity function that includes a softplus function of the feature vector and a next arrival time.

12. The system of claim 10, wherein performing event prediction uses a density function that models time probability and type probability independently.

13. The system of claim 10, wherein the transformers and the aggregation network are trained using deep learning, with a set of training data that includes synchronized time series information and timestamped event sequences.

14. The system of claim 10, wherein the aggregation network includes a stack of self-attention layers that convert outputs of the respective transformers to the feature vector.

15. The system of claim 10, wherein a hidden state of the transformer of the multivariate time series is used as a latent vector in the event prediction.

16. The system of claim 10, further determining a ranked list of past events and time series measurements that most influence the predicted event.

17. The system of claim 16, wherein determining the ranked list is performed according to attention weights from the aggregation network.

18. The system of claim 10, wherein performing the corrective action includes an action selected from the group consisting of changing a security setting for an application or hardware component, changing an operational parameter of an application or hardware component, halting and/or restarting an application, halting and/or rebooting a hardware component, changing an environmental condition, and changing a network interface's status or settings.

19. A method for performing a treatment, comprising:
measuring time series information relating to a patient;
encoding the time series information and a health event sequence for the patient using respective transformers and an aggregation network to generate a feature vector;
performing event prediction using the feature vector to identify a next health event to occur within a system; and
performing a corrective action responsive to the next health event to prevent or mitigate a negative health effect of the next health event.

20. The method of claim 19, wherein performing the corrective action includes an action selected from the group consisting of adjusting operation of a dialysis machine, adjusting dosage of an intravenously administered drug, and halting a treatment.
